(19) United States
(12) Patent Application Publication
(10) Pub. No.: US 2023/0176065 A1
(43) Pub. Date: Jun. 8, 2023

(54) METHOD OF DETECTING AN ANALYTE

(71) Applicant: Sumitomo Chemical Co., Ltd., Tokyo (JP)

(72) Inventors: Sheena Zuberi, Godmamchester (GB); Nazrul Islam, Godmamchester (GB); Jonathan Behrendt, Godmamchester (GB); Kiran Kamtekar, Godmamchester (GB)

(73) Assignee: Sumitomo Chemical Co., Ltd., Tokyo (JP)

(21) Appl. No.: 17/916,289

(22) PCT Filed: Mar. 31, 2021
(86) PCT No.: PCT/EP2021/058508

§ 371 Date: Sep. 30, 2022

(30) Foreign Application Priority Data
Mar. 31, 2020 (GB) 2004735.3

(51) Int. Cl. G01N 33/58; C09K 11/06; G01N 21/64

(52) U.S. Cl. CPC G01N 33/582; C09K 11/06; G01N 21/6428; C09K 2211/1483; G01N 2021/6439

(57) ABSTRACT

A method of identifying a target analyte in a sample containing a light-emitting marker configured to bind to the target analyte and detecting emission from the light-emitting marker. The light-emitting marker comprises a light-emitting polymer comprising a repeat unit of formula (I).

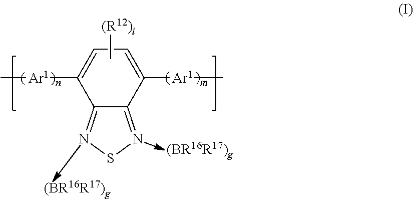

### BACKGROUND

Use of light-emitting polymers for detection of analytes is known.

GB2554666 discloses composite particles comprising silica and a light emitting polymer comprising a backbone and polar groups pendant from the backbone.

### SUMMARY

In some embodiments, the present disclosure provides a method of identifying a target analyte in a sample in which the sample is irradiated and emission from a light-emitting marker configured to bind to the target analyte, which has been added to the sample, is detected. The light-emitting marker comprises a light-emitting polymer having a first excitation wavelength and a first emission wavelength.

The light-emitting polymer comprises a repeat unit of formula (I):

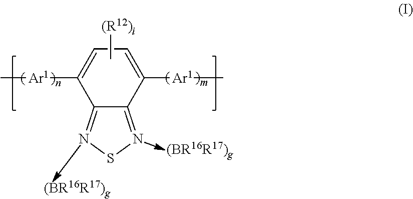

wherein i is 0, 1, or 2, n is 0, 1, 2, or 3; m is 0, 1, 2, or 3; g is 0 or 1; each BR¹⁶R¹⁷ where present may be the same or different; R¹² independently in each occurrence is a substituent; Ar¹ independently in each occurrence is an arylene group or a heteroarylene group; adjacent Ar¹ groups may be linked by a group of formula R¹⁵(R¹⁶)(R¹⁷); wherein R¹⁵ is C or B; and R¹⁶ and R¹⁷ independently in each occurrence is a substituent.

Optionally, the sample comprises one or more additional light-emitting markers, each additional light-emitting marker comprising a further light-emitting material different from the light-emitting polymer.

Optionally, each of the one or more additional light-emitting markers comprises a light-emitting material which emits light having an emission peak which is different from that of the light-emitting polymer.

Optionally, the target analyte is a target cell.

In some embodiments, the light-emitting marker is a particulate light-emitting marker dispersed in the sample. Optionally, the particulate light-emitting marker comprises the light-emitting polymer and an inorganic matrix.

In some embodiments, the light-emitting marker is dissolved in the sample.

Optionally, each R¹² is independently H, F or a polar substituent.

Optionally, each Ar¹ is independently selected from the group consisting of formulae (II)-(VIII):

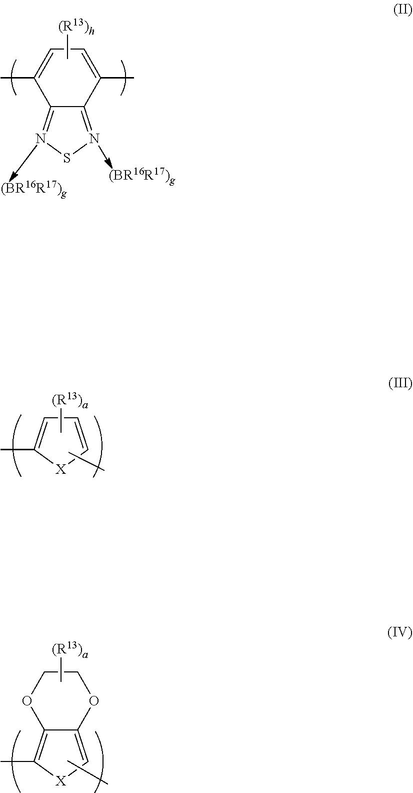

wherein R¹³ independently in each occurrence is a substituent; X is a heteroatom, preferably S; h is 0, 1, or 2, preferably 0 or 1; g is 0 or 1; a is 0, 1, or 2, preferably 0 or 1; each BR¹⁶R¹⁷ where present may be the same or different; and R¹⁶ and R¹⁷ independently in each occurrence is a substituent.

Optionally, the light-emitting polymer comprises a co-repeat unit selected from formulae (V)-(IX):

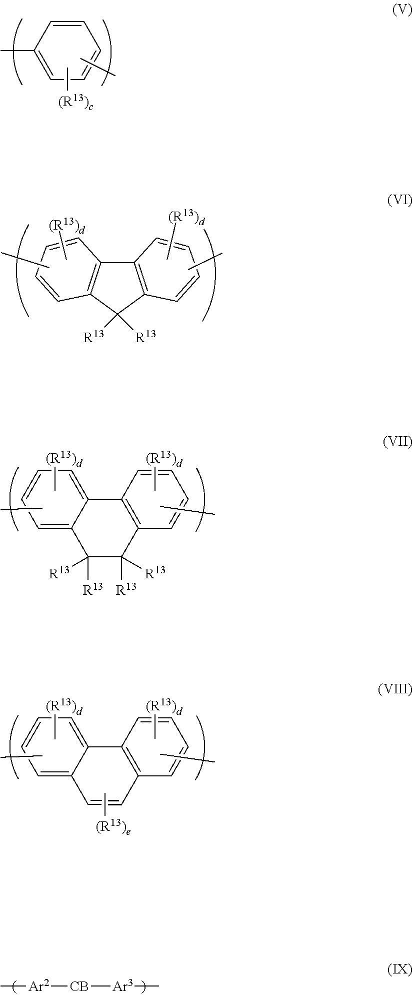

wherein R¹³ independently in each occurrence is a substituent; c is 0, 1, 2, 3 or 4; each d is independently 0, 1,2 or 3; e is 0, 1 or 2; Ar² and Ar³ each independently represent a C₆₋₂₀ arylene group or a 5-20 membered heteroarylene group which is unsubstituted or substituted with one or more substituents and CB represents a conjugation-breaking group which does not provide a conjugation path between Ar² and Ar³.

Optionally, each R¹³ is independently F or a polar substituent. Optionally, the polar substituent has formula (X):

—(Sp)ₒ—(R³)ₚ (X)

wherein Sp is a spacer group; o is 0 or 1; R³ independently in each occurrence is a polar group; p is 1 if o is 0 and p is at least 1 if o is 1.

Optionally, the light-emitting polymer comprises a repeat unit selected from formula (XI)-(XIII):

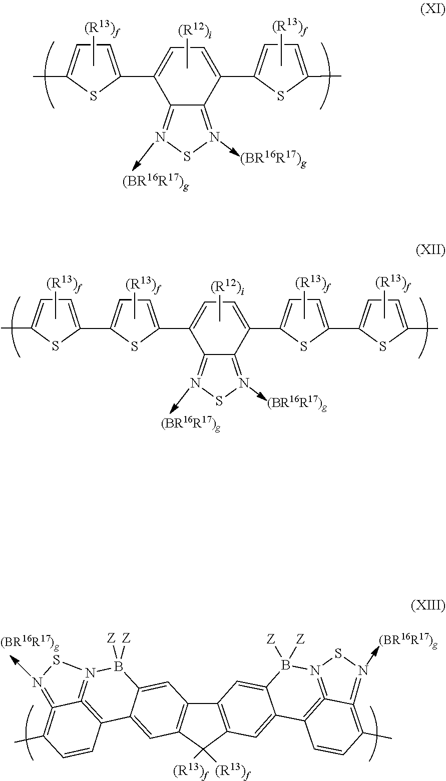

and each Z is independently selected from the group consisting of fluorine; C₁₋₂₀ amino, C₁₋₂₀ alkyl, C₃₋₂₀ cyclic group, C₆₋₂₀ aromatic group, C₆₋₂₀ heteroaromatic group, each of which is unsubstituted or substituted with one or more C₁₋₁₂ alkyl groups, C₁₋₁₂ alkoxy and/or polar substituents; electron withdrawing groups; and polar substituents.

Optionally, the light-emitting polymer comprises a repeat structure selected from formula (XIV)-(XV):

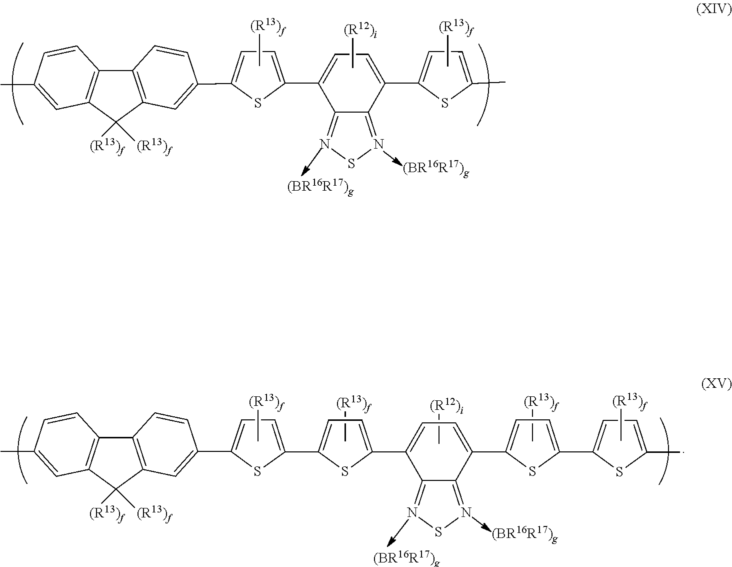

Optionally, at least one of the one or more additional light-emitting materials is a light-emitting polymer comprising a repeat unit of formula (I): Optionally, the method is a flow cytometry method.

### DESCRIPTION OF THE DRAWINGS

The disclosed technology and accompanying figures describe some implementations of the disclosed technology.

FIG. 1 illustrates a flow cytometer for use in a method according to some embodiments;

FIG. 2 shows absorption and emission spectra of a light-emitting polymer suitable for use in flow cytometry; and

FIG. 3 shows absorption spectra in water and in methanol of a light-emitting particle suitable for use in flow cytometry.

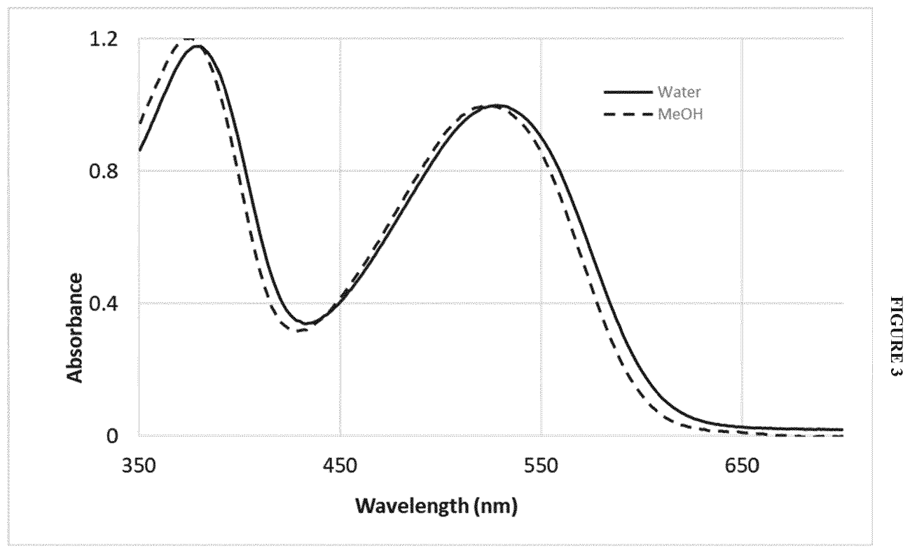

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively.

The word “or,” in reference to a list of two or more items, covers all of the following interpretations of the word: any of the items in the list, all of the items in the list, and any combination of the items in the list.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have identified certain light-emitting markers suitable for use in the detection of a target analyte, such as detection of cells in flow cytometry. The markers may have a narrow absorption band, which may allow for absorption by light from a light source having an excitation wavelength at or close to an absorption peak of the marker with little or no absorption from any other light sources having a different excitation wavelength, for example a light source intended for excitation of a different light-emitting marker. Thus, the light-emitting markers described herein (the “first light-emitting markers”) may be used in combination with one or more other light-emitting markers which contain a light-emitting material which is different from the first light-emitting markers and which absorb light at a wavelength different from the first light-emitting marker. Alternatively, two different first light-emitting markers may be used in combination.

A narrow absorption band of the light-emitting markers described herein may reduce or eliminate the need for compensation due to “cross-talk” arising from absorption by the light-emitting marker of light from a light source intended for excitation of a different first light-emitting marker, or the light-emitting material of a different light-emitting marker.

In some embodiments, the first light-emitting marker may comprise a biomolecule binding group. The biomolecule binding group may be bound, preferably covalently bound, to the light-emitting polymer. The biomolecule binding group may be provided as a side group of a repeat unit of the light-emitting polymer or as an end-group of the light-emitting polymer. In use, e.g. during flow cytometry, the light-emitting marker may be dissolved or dispersed in a sample to be analysed.

In some embodiments, the light-emitting marker may be a particulate marker comprising a matrix material and the light-emitting polymer. The matrix material is preferably an inorganic matrix material, e.g. silica. According to these embodiments, the biomolecule binding group may be bound, preferably covalently bound, to the matrix. In use, e.g. during flow cytometry, the light-emitting marker may be dispersed in a sample to be analysed.

Light-Emitting Polymer

The light-emitting polymer comprises a repeat unit of formula (I):

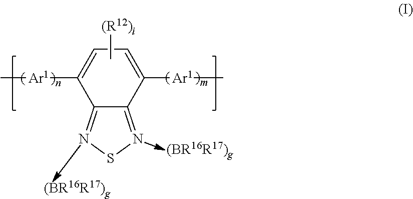

wherein i is 0, 1, or 2, n is 0, 1, 2, or 3; m is 0, 1, 2, or 3; each g is independently 0 or 1; each BR¹⁶R¹⁷ where present may be the same or different; R¹² independently in each occurrence is a substituent; Ar¹ independently in each occurrence is an arylene group or a heteroarylene group; adjacent Ar¹ groups may be linked by a group of formula R¹⁵(R¹⁶)(R¹⁷); wherein R¹⁵ is C or B; and R¹⁶ and R¹⁷ independently in each occurrence is a substituent.

In one embodiment, the light-emitting polymer consists of a repeat unit of formula (I).

R¹², where present, may be selected from the group consisting of F; C₁₋₂₀ amino; C₁₋₂₀ alkyl, C₃₋₂₀ non-aromatic cyclic group, C₆₋₂₀ aromatic group, C₅₋₂₀ heteroaromatic group, each of which is unsubstituted or substituted with one or more F, C₁₋₁₂ alkyl, C₁₋₁₂ alkoxy; electron withdrawing groups; and polar substituents.

Preferably, each R¹² is independently F or a polar substituent.

R¹⁵, where present, may be an atom selected from B or C. Each R¹⁶ and R¹⁷ may individually be selected from the group consisting of F, C₁₋₁₂ alkyl, C₃₋₂₀ cyclic group, C₆₋₂₀ aromatic group, C₆₋₂₀ heteroaromatic group, each of which is unsubstituted or substituted with one or more C₁₋₁₂ alkyl groups and/or polar substituents.

B may additionally be bonded to an atom of an adjacent Ar¹. BR¹⁶R¹⁷ may therefore be a divalent bridging group between a benzothiadiazole group and an adjacent Ar¹.

Each Ar¹ may be the same or different.

Each Ar¹ may individually be selected from the group consisting of formulae (II)-(VIII):

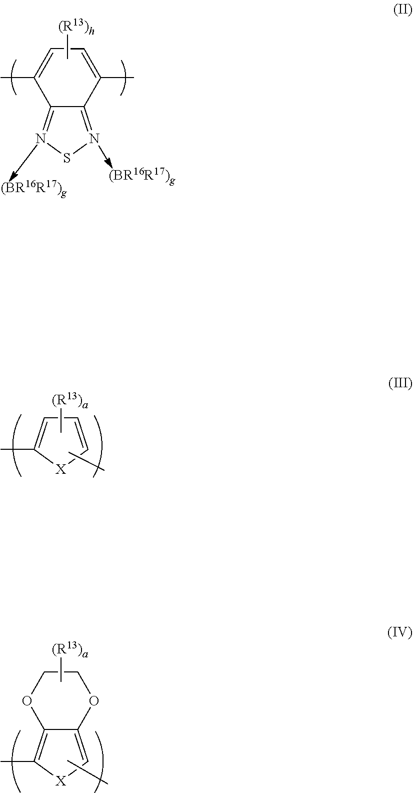

wherein R¹³ independently in each occurrence is a substituent; X is a heteroatom, preferably S; h is 0, 1, or 2, preferably 0 or 1; g is 0 or 1; a is 0, 1, or 2, preferably 0 or 1; each BR¹⁶R¹⁷where present may be the same or different; and R¹⁶ and R¹⁷ independently in each occurrence is a substituent.

B may additionally be bonded to an atom of an adjacent Ar¹. BR¹⁶R¹⁷ may therefore be a divalent bridging group between a benzothiadiazole group and an adjacent Ar¹.

The polymer may be a copolymer comprising one or more co-repeat units. Co-repeat units include, without limitation, C₆₋₂₀ arylene repeat units; C5-20 arylene repeat units; and conjugation-breaking repeat units. A conjugation-breaking repeat unit as described herein is a repeat unit which does not provide a path of alternating C-C single bonds and C═C double bonds between repeat units directly linked to the conjugation braking repeat unit.

In one embodiment, the light-emitting polymer may comprise a co-repeat unit selected from the group consisting of formulae (V)-(VIII):

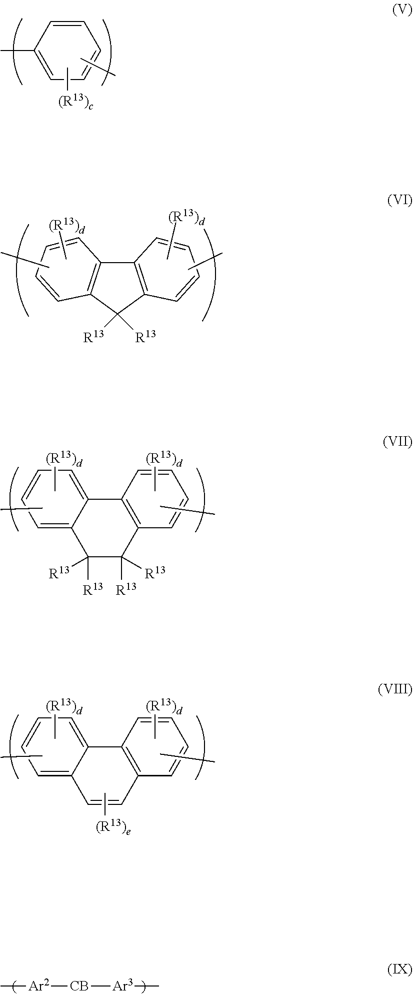

wherein R¹³ independently in each occurrence is a substituent; c is 0, 1, 2, 3 or 4, preferably 1 or 2; each d is independently 0, 1,2 or 3, preferably 0 or 1; e is 0, 1 or 2, preferably 2; Ar² and Ar³ each independently represent a C₆₋₂₀ arylene group or a 5-20 membered heteroarylene group which is unsubstituted or substituted with one or more substituents and CB represents a conjugation-breaking group which does not provide a conjugation path between Ar² and Ar³.

Optionally, Ar² and Ar³ are each independently phenylene which is unsubstituted or substituted with one or more substituents, optionally one or more substituents R¹³.

Optionally, CB contains at least one sp³ hybridised carbon atom separating Ar² and Ar³.

Optionally, CB is a C₁₋₂₀ branched or linear alkylene group wherein one or more H atoms may be replaced with F and one or more non-adjacent C atoms of the alkylene group may be replaced with O, S, CO, COO or Si(R⁷)₂ wherein R⁷ in each occurrence is independently a C₁₋₂₀ hydrocarbyl group.

R¹³, where present, may be selected from the group consisting of F; C₁₋₂₀ amino, C₁₋₂₀ alkyl, C₃₋₂₀ cyclic group, C₆₋₂₀ aromatic group, C₆₋₂₀ heteroaromatic group, each of which is unsubstituted or substituted with one or more F, C₁₋₁₂ alkyl groups, C₁₋₁₂ alkoxy; electron withdrawing groups; and polar substituents.

Preferably, where present, each R¹³ is independently F or a polar substituent.

Each R¹⁶ and R¹⁷ may individually be selected from the group consisting of F, C₁₋₁₂ alkyl, C₃₋₂₀ cyclic group, C₆₋₂₀ aromatic group, C₆₋₂₀ heteroaromatic group, each of which is unsubstituted or substituted with one or more C₁₋₁₂ alkyl groups and/or polar substituents.

Preferably, a polar substituent as described herein has formula (X):

—(Sp)ₒ—(R³)ₚ (X)

wherein Sp is a spacer group; o is 0 or 1; R³ independently in each occurrence is a polar group; p is 1 if o is 0 and p is at least 1, optionally 1, 2, 3 or 4, if o is 1.

Preferably, Sp is selected from:
C₁₋₂₀ alkylene or phenylene-C₁₋₂₀ alkylene wherein one or more non-adjacent C atoms may be replaced with O, S, N or C═O;
a C₆₋₂₀ arylene or 5-20 membered heteroarylene, more preferably phenylene, which, other than the one or more polar groups R³, may be unsubstituted or substituted with one or more non-polar substituents, optionally one or more C₁₋₁₂ alkyl groups.

More preferably, Sp is selected from:
C₁₋₂₀ alkylene wherein one or more non-adjacent C atoms may be replaced with O, S or CO; and
a C₆₋₂₀ arylene or a 5-20 membered heteroarylene, even more preferably phenylene, which may be unsubstituted or substituted with one or more non-polar substituents.

R³ may be an ionic group or a non-ionic polar group.

An exemplary non-ionic polar group has formula —O(R⁴O)ᵥ—R⁵ wherein R⁴ in each occurrence is a C₁₋₁₀ alkylene group, optionally a C₁₋₅ alkylene group, wherein one or more non-adjacent, non-terminal C atoms of the alkylene group may be replaced with O, R⁵ is H or C₁₋₅ alkyl, and v is 0 or a positive integer, optionally 1-10. Preferably, v is at least 2. More preferably, v is 2 to 5. The value of v may be the same in all the polar groups of formula —O(R⁴O)ᵥ—R⁵. The value of v may differ between polar groups of the same polymer.

Optionally, the non-ionic polar group has formula O(CH₂CH₂O)ᵥR⁵ wherein v is at least 1, optionally 1-10 and R⁵ is a C₁₋₅ alkyl group, preferably methyl. Preferably, v is at least 2. More preferably, v is 2 to 5, most preferably v is 3.

By “non-terminal C atom” of an alkyl group as used herein means a C atom other than the methyl group at the end of an n-alkyl group or the methyl groups at the ends of a branched alkyl chain.

An ionic group R³ may be anionic or cationic.

Exemplary anionic group are —COO⁻, a sulfonate group; hydroxide; sulfate; phosphate; phosphinate; or phosphonate.

An exemplary cationic group is —N(R6⁵)₃⁺ wherein R⁶ in each occurrence is H or C₁₋₁₂ hydrocarbyl. Preferably, each R⁵ is a C₁₋₁₂ hydrocarbyl.

A light-emitting polymer comprising cationic or anionic groups comprises counterions to balance the charge of these ionic groups. An anionic or cationic group and counterion may have the same valency, with a counterion balancing the charge of each anionic or cationic group. The anionic or cationic group may be monovalent or polyvalent. Preferably, the anionic and cationic groups are monovalent.

The light-emitting polymer may comprise a plurality of anionic or cationic polar groups wherein the charge of two or more anionic or cationic groups is balanced by a single counterion. Optionally, the polar groups comprise anionic or cationic groups comprising di-or trivalent counterions.

In the case of an anionic group, the cation counterion is optionally a metal cation, optionally Li⁺, Na⁺, K⁺, Cs⁺, preferably Cs⁺, or an organic cation, optionally ammonium, such as tetraalkylammonium, ethylmethyl imidazolium or pyridinium.

In the case of a cationic group, the anion counterion is optionally a halide; a sulfonate group, optionally mesylate or tosylate; hydroxide; carboxylate; sulfate; phosphate; phosphinate; phosphonate; or borate.

In some embodiments, the light-emitting polymer comprises polar groups selected from groups of formula —O(R⁴O)ᵥ—R⁵ and/or ionic groups. Preferably, the light-emitting polymer comprises polar groups selected from groups of formula —O(CH₂CH₂O)ᵥR⁵ and/or anionic groups of formula —COO⁻.

Preferably, the light-emitting polymer comprises a repeat unit of formula (XI)-(XIII):

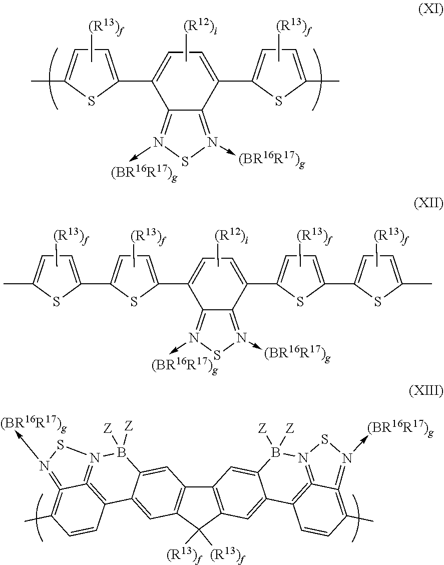

wherein R¹², R¹³, R¹⁶, R¹⁷, and g are defined as above; and each Z is independently selected from the group consisting of fluorine; C₁₋₂₀ amino, C₁₋₁₂ alkyl, C₃₋₂₀ cyclic group, C₆₋₂₀ aromatic group, C₆₋₂₀ heteroaromatic group, each of which is unsubstituted or substituted with one or more C₁₋₁₂ alkyl groups, C₁₋₁₂ alkoxy and/or polar substituents; electron withdrawing groups; and polar substituents.

Preferably, the light-emitting polymer comprises a repeat structure of formula (XIV)-(XV):

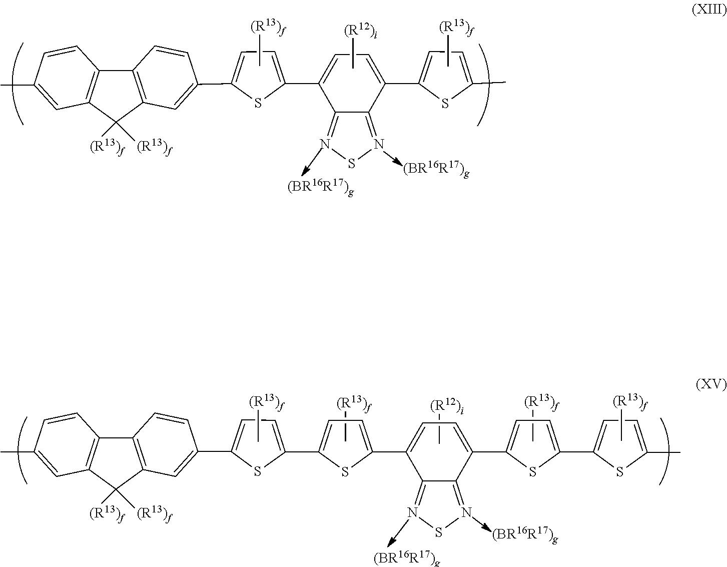

wherein R¹², R¹³, R¹⁶, R¹⁷, f and g are defined as above.

In one embodiment, the light-emitting polymer consists of a repeat unit selected formula (XI)-(XIII).

In one embodiment, the light-emitting polymer consists of a repeat structure selected formula (XIV)-(XV).

The marker may be a tag and bind to the analyte. The light-emitting marker may further comprise a biomolecule binding group.

The polymer preferably has a solubility in water or a C₁₋₆ alcohol, preferably methanol, at 20° C. of at least 0.1 mg/ml.

Solubility in water or an alcohol may be provided by polar substituents of a repeat unit of formula (I) and/or one or more co-repeat units, e.g. polar groups of formula (II).

The polymer preferably has an absorption peak at a wavelength greater than 500 nm, preferably in the range of 500-750 nm.

Mechanisms for energy transfer include, for example, resonant energy transfer; Forster (or fluorescence) resonance energy transfer (FRET), quantum charge exchange (Dexter energy transfer) and the like.

Unless stated otherwise, emission spectra of light-emitting materials or markers as described herein are as measured in water, using a Hamamatsu C9920-02 instrument having a set up wavelength 300 nm-950 nm; light source 150 W xenon light and bandwidth 10 nm or less (FWHM). Initially the system is calibrated with red (395 nm), green (375 nm) and blue (335 nm) glass standards. Two 5 ml long necked cuvettes (one filled with reference solvent i.e. water) and one filled with a sample of 1 mg/ml is diluted 1 in 100 for a dissolved light-emitting marker or 1 mg/ml diluted ˜1 in 10 with water for a particulate light-emitting marker. The final concentration of the sample is altered to obtain a transmission data in the range 0.25-0.35. An average of 3 measurements for each sample is recorded.

Unless stated otherwise, absorption spectra of light-emitting materials or markers as described herein are measured in water using a Cary 5000 UV-VIS-NIR Spectrometer. Measurements are taken from 175 nm to 3300 nm using a PbSmart NIR detector for extended photometric range with variable slit widths (down to 0.01 nm) for optimum control over data resolution. A baseline run with water in front and back 5 ml matched cuvettes (600 to 250 nm) following which the back cuvette reference remains as water and the front cuvette is changed to a sample of 1 mg/ml diluted 1 in 100 for a dissolved light-emitting marker or 1 mg/ml diluted ˜1 in 10 with water for a particulate light-emitting marker.

Conjugated light-emitting polymers as described herein may be formed by polymerising monomers comprising leaving groups that leave upon polymerisation of the monomers to form conjugated repeat units. Exemplary polymerization methods include, without limitation, Yamamoto polymerization as described in, for example, T. Yamamoto, “Electrically Conducting And Thermally Stable pi-Conjugated Poly(arylene)s Prepared by Organometallic Processes”, Progress in Polymer Science 1993, 17, 1153-1205, the contents of which are incorporated herein by reference and Suzuki polymerization as described in, for example, WO 00/53656, WO 2003/035796, and U.S. Pat. No. 5,777,070, the contents of which are incorporated herein by reference.

Light-Emitting Particle

The light-emitting marker may be in the form of a light-emitting particle comprising or consisting of the light-emitting material.

In some embodiments, formation of a light-emitting nanoparticle comprising or consisting of a polymer as described herein may include collapse of the light-emitting polymer.

In some embodiments, the particle may have a particulate core and, optionally, a shell wherein at least one of the core and shell contains the light-emitting material. Preferably, the light-emitting particle contains the light-emitting material and a matrix material. Matrix materials include, without limitation, inorganic matrix materials, optionally inorganic oxides, optionally silica. The matrix may at least partially isolate the light-emitting material from the surrounding environment. This may limit any effect that the external environment may have on the lifetime of the light-emitting material.

The light-emitting polymer may be mixed with the matrix material.

The light-emitting polymer may be bound, e.g. covalently bound, to the matrix material.

Polymer chains of the light-emitting polymer may extend across some or all of the thickness of the core and/or shell. Polymer chains may be contained within the core and/or shell or may protrude through the surface of the core and/or shell.

In some embodiments, the particle core may be formed by polymerisation of a silica monomer in the presence of the light-emitting polymer, for example as described in WO 2018/060722, the contents of which are incorporated herein by reference.

In some embodiments, the particle core comprises a core which comprises or consists of the light-emitting polymer and at least one shell surrounding the inner core. The at least one shell may be silica.

Optionally, at least 0.1 wt % of total weight of the particle core consists of the light-emitting polymer. Preferably at least 1, 10, 25 wt % of the total weight of the particle core consists of the light-emitting polymer.

Optionally at least 50 wt % of the total weight of the particle core consists of the matrix material. Preferably at least 60, 70, 80, 90, 95, 98, 99, 99.5, 99.9 wt % of the total weight of the particle core consists of the matrix material.

The particle core as described herein is the light-emitting particle without any surface groups, e.g. binding groups or solubilising groups, thereon.

In one embodiment of the present disclosure, at least 70 wt % of the total weight of the particle core consists of the light-emitting material or materials and silica. Preferably at least 80, 90, 95, 98, 99, 99.5, 99.9 wt % of the total weight of the particle core consists of the light-emitting material and silica. More preferably the particle core consists essentially of the light-emitting material and silica.

Preferably, the particles have a number average diameter of no more than 5000 nm, more preferably no more than 2500 nm, 1000 nm, 900 nm, 800 nm, 700 nm, 600 nm, 500 nm or 400 nm as measured by dynamic light scattering (DLS) using a Malvern Zetasizer Nano ZS.

Preferably the particles have a number average diameter of between 5-5000 nm, optionally 10-1000 nm, preferably between 10-500 nm, most preferably between 10-100 nm as measured by a Malvern Zetasizer Nano ZS.

Light-emitting particles as described herein may be provided as a colloidal suspension comprising the particles suspended in a liquid. Preferably, the liquid is selected from water, C₁₋₁₀ alcohols and mixtures thereof. Preferably, the particles form a uniform (non-aggregated) colloid in the liquid. In some embodiments, each of the first, second and any further light-emitting polymers are light-emitting particles dispersed in the liquid. In some embodiments, one or more of the light-emitting polymers is in particle form dispersed in the liquid and one or more of the light-emitting polymers is dissolved in the liquid.

The liquid may be a solution comprising salts dissolved therein, optionally a buffer solution.

In some embodiments, the particles may be stored in a powder form, optionally in a lyophilised or frozen form.

Biomolecule Binding Groups

The biomolecule binding group of a light-emitting marker may be selected according to a target analyte of a sample to be analysed.

In the case of a light-emitting particle, the biomolecule binding group may be bound to a surface of the particle core, e.g. bound to a matrix material of the light-emitting particle core. Each biomolecule binding group may be directly bound to the surface of a light-emitting particle core or may be spaced apart therefrom by one or more surface binding groups. The surface binding group may comprise polar groups. Optionally, the surface binding group comprises a polyether chain. By “polyether chain” as used herein is meant a chain having two or more ether oxygen atoms.

The surface of a light-emitting particle core may be reacted to form a group at the surface capable of attaching to a biomolecule binding group. Optionally, a silica-containing particle is reacted with a siloxane.

Biomolecule binding groups may be selected from the group consisting of: DNA, RNA, peptides, carbohydrates, antibodies, antigens, enzymes, proteins and hormones.

A preferred biomolecule binding group comprises or consists of biotin. In some embodiments, a biotin biomolecule binding group binds directly to a target analyte. In some embodiments, biomolecule binding group comprises biotin bound to a protein having a plurality of biotin binding sites, preferably streptavidin, neutravidin, avidin or a recombinant variant or derivative thereof and biotinylated biomolecule having a second biotin group is bound to the same protein. The biotinylated biomolecule may be selected according to the target analyte. The biotinylated biomolecule may comprise an antigen binding fragment, e.g. an antibody, which may be selected according to a target antigen.

In the case of a light-emitting particle marker, the biomolecule binding group may be bound to a surface of the particle core, e.g. bound to a matrix material of the light-emitting particle. Each biomolecule binding group may be directly bound to the surface of a light-emitting particle or may be spaced apart therefrom by one or more surface binding groups. The surface binding group may comprise polar groups. Optionally, the surface binding group comprises a polyether chain. By “polyether chain” as used herein is meant a chain having two or more ether oxygen atoms.

The surface of a light-emitting particle core may be reacted to form a group at the surface capable of attaching to a functional group. Optionally, a silica-containing particle is reacted with a siloxane.

Flow Cytometry

Light-emitting markers as described herein may be used as luminescent probes for detecting or labelling a biomolecule or a cell. In some embodiments, the particles may be used as a luminescent probe in an immunoassay such as a lateral flow or solid state immunoassay. Optionally the particles are for use in fluorescence microscopy, flow cytometry, next generation sequencing, in-vivo imaging, or any other application where a light-emitting marker is brought into contact with a sample to be analysed. The analysis may be performed using time-resolved spectroscopy. The applications can medical, veterinary, agricultural or environmental applications whether involving patients (where applicable) or for research purposes.

In use, the light-emitting markers may bind to target biomolecules which include without limitation DNA, RNA, peptides, carbohydrates, antibodies, antigens, enzymes, proteins and hormones. The target biomolecule may or may not be a biomolecule, e.g. a protein, at a surface of a cell. The binding group of the light-emitting markers may bind to target biomolecules.

A sample to be analysed may be brought into contact with the particles, for example the particles in a colloidal suspension.

In some embodiments, the sample is analysed by flow cytometry. In flow cytometry, the light-emitting markers are irradiated by at least one wavelength of light, optionally two or more different wavelengths, e.g. one or more wavelengths including at least one of about 355, 405, 488, 530, 561 and 640 nm, each of which may be ±10 nm. Light emitted by the light-emitting markers may be collected by one or more detectors. To provide a background signal for calculation of a staining index, measurement may be made of particles mixed with cells which do not bind to the particles.

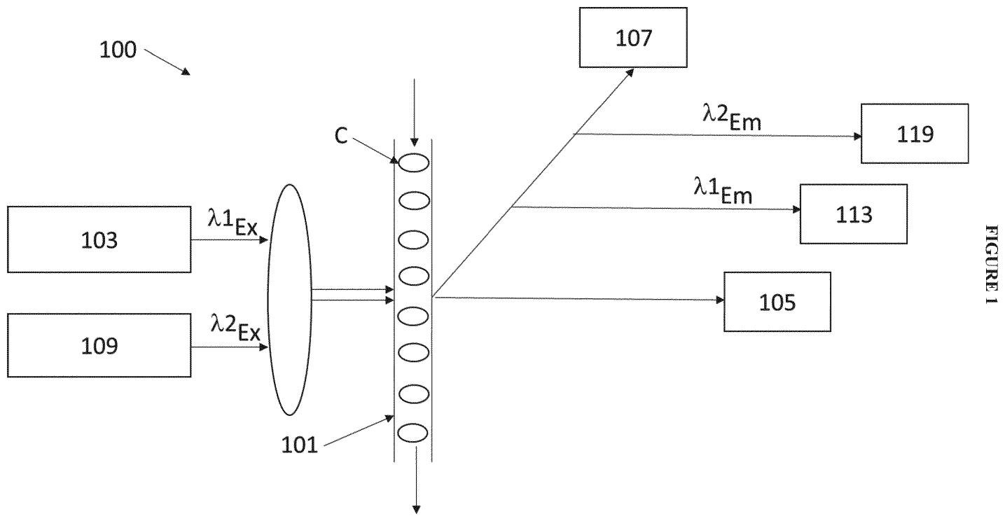

FIG. 1 schematically illustrates flow cytometer 100.

The flow cytometer comprises a flow channel 101 through which cells may pass in a single file; a first light source 103, e.g. a laser, configured to irradiate the flow channel with light of a first excitation wavelength λ1Ex; a forward scatter detector 105; a side scatter detector 107; and a first photodetector 113 configured to detect light of a first emission wavelength λ1Em emitted from a first light-emitting marker bound to a cell upon excitation by the first excitation wavelength λ1Ex.

The apparatus may further comprise at least one further light source 105, e.g. a laser, configured to irradiate the flow channel with light of a second excitation wavelength λ2Ex and a photodetector configured to detect light of a second emission wavelength λ2Em emitted from a further light-emitting marker bound to a cell upon excitation by the second excitation wavelength λ2Ex. λ1Ex and λ2Ex are different. λ1Em and λ2Em are different.

Signals received by the forward scatter detector, side scatter detector and photodetectors may be transmitted by wired or wireless transmission to a signal processor (not shown).

### EXAMPLES

### Polymer Example 1

A polymer having the following structure was prepared by Suzuki polymerisation, in which the fluorene repeat units were formed by polymerisation of ethyl ester monomers followed by hydrolysis of the ester group as described in WO 2012/133229, the contents of which are incorporated herein by reference:

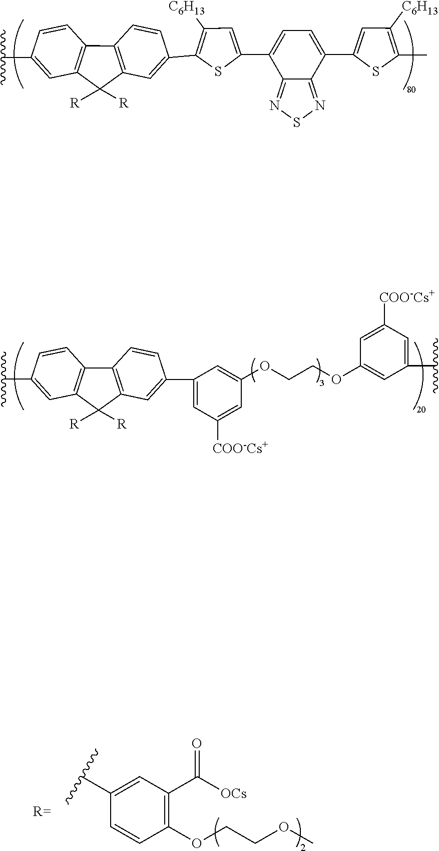

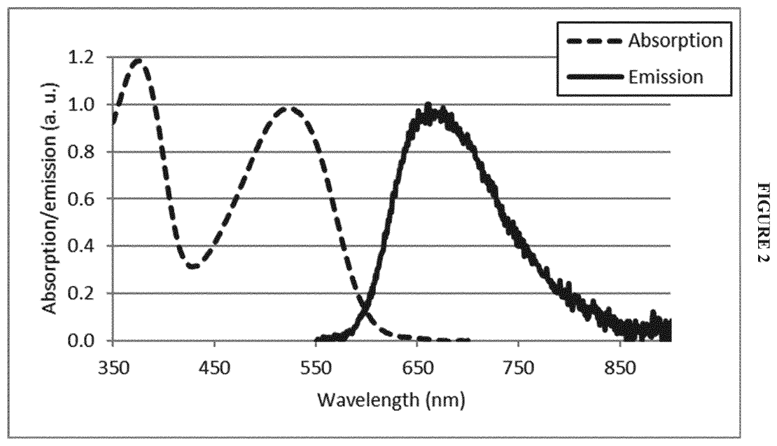

Absorption and emission spectra of Polymer Example 1 in methanol solution are shown in FIG. 2. As shown in FIG. 2, the absorption peak is close to 530 nm, making this polymer well matched to use in analytical apparatus, e.g. a flow cytometer, having a light source that emits at about 530 nm.

### Particle Example 1

Silica nanoparticles having chains of Polymer Example 1 were formed by polymerisation of tetraethylorthosilicate (TEOS) as described in GB 2554666, the contents of which are incorporated herein, in a methanol solution containing Polymer Example 1 dissolved therein.

Absorption spectra of the particles in methanol and in water are shown in FIG. 2.

1. A method of identifying a target analyte in a sample, the method comprising: irradiating a sample comprising a light-emitting marker configured to bind to the target analyte and comprising a light-emitting polymer having a first excitation wavelength and a first emission wavelength; and detecting emission from the light-emitting marker, wherein the light-emitting polymer comprises a repeat unit of formula (I): 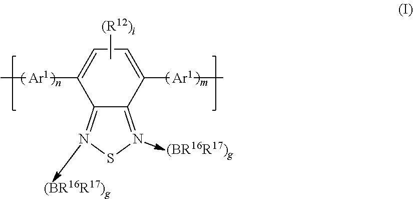
wherein i is 0, 1, or 2, n is 0, 1, 2, or 3; m is 0, 1, 2, or 3; g is 0 or 1; each BR¹⁶R¹⁷ where present may be the same or different; R¹² independently in each occurrence is a substituent; Ar¹ independently in each occurrence is an arylene group or a heteroarylene group; adjacent Ar¹ groups may be linked by a group of formula R¹⁵(R¹⁶)(R¹⁷); wherein R¹⁵ is C or B; and R¹⁶ and R¹⁷ independently in each occurrence is a substituent.

2. A method according to claim 1, wherein the sample comprises one or more additional light-emitting markers, each comprising a further light-emitting material different from the light-emitting polymer according to claim 1.

3. A method according to claim 2, wherein each of the one or more additional light-emitting markers comprises a light-emitting material which emits light having an emission peak which is different from that of the light-emitting polymer.

4. A method according to claim 1, wherein the target analyte is a target cell.

5. The method according to claim 1, wherein the light-emitting marker is a particulate light-emitting marker dispersed in the sample.

6. The method according to claim 5, wherein the particulate light-emitting marker comprises the light-emitting polymer according to claim 5 and an inorganic matrix.

7. The method according to claim 6 wherein the inorganic matrix is silica.

8. The method according to claim 1, wherein the light-emitting marker is dissolved in the sample.

9. The method according to claim 1, wherein each R¹² is independently H, F or a polar substituent.

10. The method according to claim 1, wherein each Ar¹ is independently selected from the group consisting of formulae (II)-(VIII): 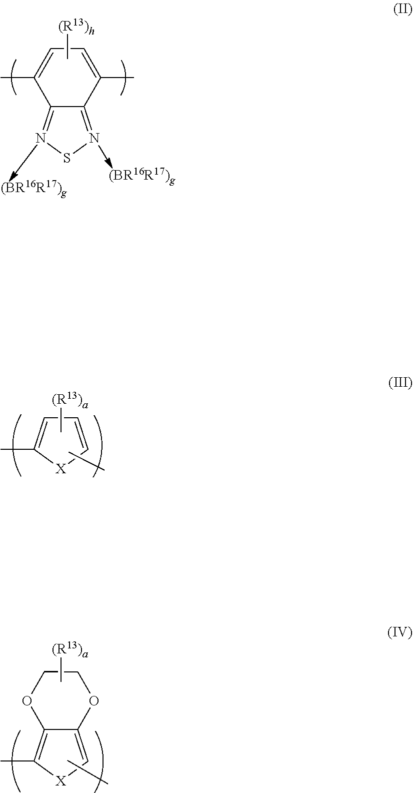
wherein R¹³ independently in each occurrence is a substituent; X is a heteroatom; h is 0, 1, or 2; g is 0 or 1; a is 0, 1, or 2; each BR¹⁶R¹⁷ where present may be the same or different; and R¹⁶ and R¹⁷ independently in each occurrence is a substituent.

11. The method according to claim 9 wherein each X is S.

12. The method according to claim 1, wherein the light-emitting polymer comprises a co-repeat unit selected from formulae (V)-(IX): 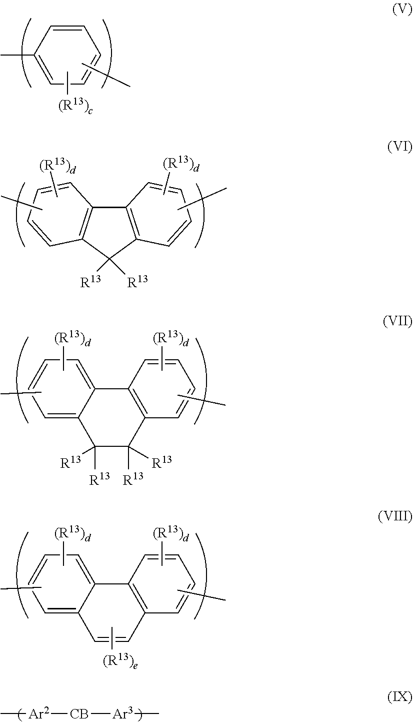
wherein R¹³ independently in each occurrence is a substituent; c is 0, 1, 2, 3 or 4; each d is independently 0, 1,2 or 3; e is 0, 1 or 2; Ar² and Ar³ each independently represent a C₆₋₂₀ arylene group or a 5-20 membered heteroarylene group which is unsubstituted or substituted with one or more substituents and CB represents a conjugation-breaking group which does not provide a conjugation path between Ar² and Ar³.

13. The method according to claim 10, wherein each R¹³ is independently F or a polar substituent.

14. The method according to claim 13, wherein the polar substituent has formula (X):
—(Sp)ₒ—(R³)ₚ (X)
wherein Sp is a spacer group; o is 0 or 1; R³ independently in each occurrence is a polar group; p is 1 if o is 0 and p is at least 1 if o is 1.

15. The method according to claim 14 wherein the polar substituent of formula (X) comprises at least one ionic group R³.

16. The method according to claim 1, wherein the light-emitting polymer comprises a repeat unit selected from formula (XI)-(XIII): 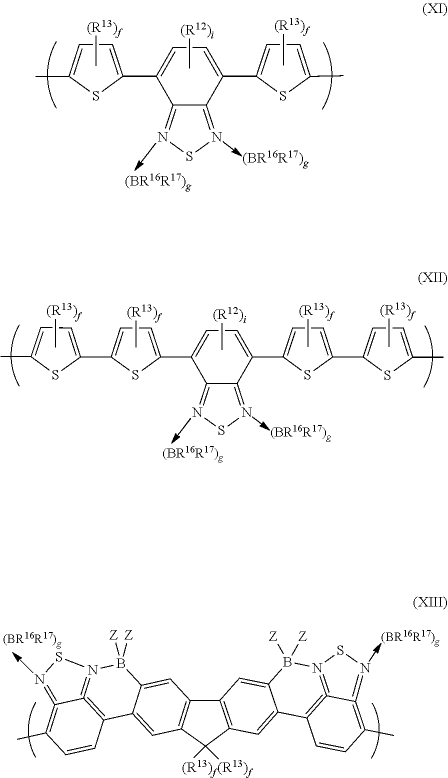
and each Z is independently selected from the group consisting of fluorine; C₁₋₂₀ amino, C₁₋₂₀ alkyl, C₃₋₂₀ on cyclic group, C₆₋₂₀ aromatic group, C₆₋₂₀ heteroaromatic group, each of which is unsubstituted or substituted with one or more C₁₋₁₂ alkyl groups, C₁₋₁₂ alkoxy and/or polar substituents; electron withdrawing groups; and polar substituents.

17. The method according to claim 16, wherein the light-emitting polymer comprises a repeat structure selected from formula (XIV)-(XV):

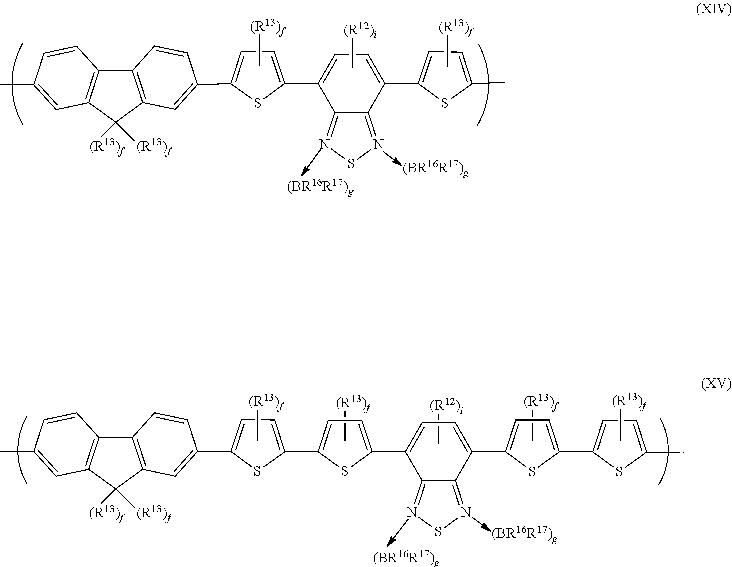

18. The method according to claim 2, wherein at least one of the one or more additional light-emitting materials is a light-emitting polymer comprising a repeat unit of formula (I):

19. The method according to claim 1 wherein the method is a flow cytometry method.
